(19) United States
(12) Patent Application Publication
(10) Pub. No.: US 2008/0287756 A1
(43) Pub. Date: Nov. 20, 2008

(54) Pulse oximetry relational alarm system for early recognition of instability and catastrophic occurrences

(76) Inventor: Lawrence A. Lynn, Columbus, OH (US)

Correspondence Address: NELLCOR PURITAN BENNETT LLC;ATTN: IP LEGAL, 60 Middletown Avenue, North Haven, CT, 06473, US

(21) Appl. No.: 12/152,747

(22) Filed: May 16, 2008

Related U.S. Application Data
(63) Continuation of application No. 11/148,325, filed on Jun. 9, 2005, now Pat. No. 7,398,115.
Application No. 11/148,325 is a continuation of application No. 10/162,466, filed on Jun. 3, 2002, now abandoned.
Application No. 11/148,325 is a continuation-in-part of application No. 10/150,582, filed on May 17, 2002, now Pat. No. 7,081,095.
Application No. 10/162,466 is a continuation-in-part of application No. 10/150,582, filed on May 17, 2002, now Pat. No. 7,081,095.
Application No. 11/148,325 is a continuation-in-part of application No. 10/150,842, filed on May 17, 2002.
Application No. 10/162,466 is a continuation-in-part of application No. 10/150,842, filed on May 17, 2002.
Application No. 10/162,466 is a continuation-in-part of application No. 10/132,535, filed on Apr. 24, 2002, now Pat. No. 6,748,252.
Application No. 10/132,535 is a division of application No. 09/776,771, filed on Feb. 6, 2001, now Pat. No. 6,760,608.
Application No. 09/776,771 is a continuation of application No. 09/115,226, filed on Jul. 14, 1998, now Pat. No. 6,223,064.
(60) Provisional application No. 60/295,484, filed on Jun. 1, 2001.
(60) Provisional application No. 60/291,691, filed on May 17, 2001.
(60) Provisional application No. 60/291,687, filed on May 17, 2001.
(60) Provisional application No. 60/291,691, filed on May 17, 2001.
(60) Provisional application No. 60/291,687, filed on May 17, 2001.
(60) Provisional application No. 60/291,691, filed on May 17, 2001.
(60) Provisional application No. 60/291,687, filed on May 17, 2001.
(60) Provisional application No. 60/291,691, filed on May 17, 2001.
(60) Provisional application No. 60/291,687, filed on May 17, 2001.
(60) Provisional application No. 60/052,438, filed on Jul. 14, 1997.
(60) Provisional application No. 60/052,439, filed on Jul. 14, 1997.

(51) Int. Cl. A61B 5/00

(52) U.S. Cl. 600/323

(57) ABSTRACT

A relational pulse oximetry alarm system and method is presented for earlier identification of the occurrence of an adverse clinical event. The system includes a pulse oximeter based microprocessor alarm system which provides an alarm based on a relational conformation of a plurality of time series and further based on the recognition of specific dynamic patterns of interaction between a plurality of corresponding and related time series including the occurrence of pathophysiologic divergence of two or more time series outputs. The processor is programmed to compare a first time series to a second time series to produce a comparison result, to identify a relationship between the first time series and the second time series, to identify a relational threshold breach, and to output an alarm based on the relational threshold breach. The system can include an oximeter testing system for predicting the timeliness of the response of the alarm of a pulse oximeter to the occurrence of an adverse clinical event.

This application is a continuation of U.S. application Ser. No. 11/148,325, filed Jun. 9, 2005, which is a continuation of U.S. application Ser. No. 10/162,466, filed Jun. 3, 2002, now abandoned, which claims the benefit of provisional application No. 60/295,484, filed Jun. 1, 2002, each of which is incorporated by reference in its entirety as if completely disclosed herein. Ser. No. 11/148,325, and Ser. No. 10/162,466 are continuations-in-part of application Ser. No. 10/150,582, filed May 17, 2002, now U.S. Pat. No. 7,081,095, which is incorporated herein by reference, and which claims the benefit of provisional applications Nos. 60/291,687 and 60/291,691 filed on May 17, 2001 (both of which are also incorporated herein by reference). The present application Ser. No. 11/148,325, and Ser. No. 10/162,466 are also continuations-in-part of U.S. application Ser. No. 10/150,842, filed May 17, 2002 (which is incorporated herein by reference), currently pending, which also claims the benefit of aforesaid provisional applications Nos. 60/291,687 and 60/291,691. Application Ser. No. 10/162,466 is also a continuation-in-part of U.S. application Ser. No. 10/132,535, filed Apr. 24, 2002, now U.S. Pat. No. 6,748,252, which is a division of U.S. application Ser. No. 09/776,771, filed Feb. 6, 2001, now U.S. Pat. No. 6,760,608, which is a continuation of U.S. application Ser. No. 09/115,226, filed Jul. 14, 1998, now U.S. Pat. No. 6,223,064, which claims the benefit of provisional applications Nos. 60/052,438 and 60/052,439, filed on Jul. 14, 1997. Each of these references is incorporated herein by reference.

### FIELD OF THE INVENTION

This application relates to improved alarm systems for oximetry and to methods of relational signal processing to enhance the specificity and timeliness of the alarms of pulse oximeters.

### BACKGROUND AND SUMMARY OF THE INVENTION

Delay in recognition of respiratory instability and/or arrest is a very common issue in many hospitals. Many serious diseases progress to respiratory arrest and this progression can occur very suddenly. Accordingly, it is desirable to recognize respiratory arrest early. The period of respiratory instability preceding respiratory arrest is highly variable ranging from more than 24 hours to less than a minute. Respiratory instability is painless and generally causes shortness of breath, which is often discounted by hospital personnel since this symptom is so common for patients in the hospital. For these reasons the significance of the warning symptoms and signs of impending respiratory arrest are often missed by physicians and nurses since the symptoms are thought to simply be due to, for example, anxiety, postoperative pain, or fever.

In many cases respiratory arrest can be readily reversed and the patient stabilized by a simple bag and mask or even simple mouth to mouth resuscitation. However, once respiratory arrest induces cardiac arrest chest compressions and cardiopulmonary resuscitation becomes necessary and the success of such resuscitation in this setting is very low. The progression of respiratory arrest to cardiac arrest is perhaps best termed as a state of “dual (oxygen depletion) arrest”.

To compare the significance of dual arrest it is important to contrast the process of sudden cardiac arrest as the primary event with that of a sudden primary respiratory arrest. In contrast with the dual arrest state described above, oxygen stores in arteries, veins, and lungs are retained after a sudden cardiac arrest. If the heart can be restarted (which can often be achieved within seconds by defibrillation) these oxygen stores are immediately available to restore oxygen to the brain and heart muscle and normal ventilation generally spontaneously returns. If on the other hand a respiratory arrest is the initial event, the body oxygen stores are depleted as the heart pumps the remaining oxygen stores to keep the brain and heart muscle alive after the cessation of breathing. (The heart rate generally slows during this period to reduce the use of oxygen by the heart muscle). When the oxygen stores are depleted the heart muscle stops contracting or cardiac electrical instability develops producing a secondary cardiac arrest, the state of dual arrest. Upon progression to the state of dual arrest, both heart and lungs must be restarted, which can be difficult.

Patients who are pregnant, obese, or have heart or lung disease may have lower oxygen stores at the time of the respiratory arrest. This means that depletion and progression to dual arrest can occur very rapidly in these patients. To understand the critical limitation of time between the onset of respiratory arrest and the development of dual arrest due to oxygen depletion in the real world, consider the case of respiratory arrest (as, for example, due to an adverse drug reaction) of a mother at near term pregnancy. After the respiratory arrest both the baby and the mother are rapidly depleting the mother's oxygen stores (which is already low due to the reduced size of the lungs due to size of the full term baby). Both the low oxygen stores and the more rapid depletion of those stores can greatly shorten the time to dual arrest. Here it can be seen that the time between the onset of the respiratory arrest and the sounding of the alarm may be pivotal toward determining whether simple ventilation is appropriate or complex CPR should be employed.

Today, telemetry and bedside monitors with both pulse oximetry and EKG are in wide use on hospital wards as early warning devices. Unfortunately while the alarm systems of the EKG components of the multimode monitors are excellent for immediately identifying cardiac arrest, the alarms of the incorporated pulse oximeters may not be suited for early identification of respiratory arrest.

The occurrence of important delays in oximeter detection of critical clinical events has been known for many years (see “Delayed detection of hypoxic events by pulse oximeters: computer simulations”, Verhoeff F, Sykes M K. Anaesthesia February 1990; 45(2):103-9). One of the factors contributing to this delay has been the high false alarm rate of pulse oximeters. Pulse oximeters have traditionally produced an output, which can be affected by motion and other sources of artifact. U.S. Pat. No. 6,206,830 entitled “signal processing apparatus and method”, the contents of which is incorporated by reference as if completely disclosed herein, provides background for some of the deficiencies associated with the present art of monitoring patients using pulse oximetry. To address these deficiencies, in the present art, the output signal is subjected to a wide range of signal processing including different filters such as low pass and averaging filters as well as adaptive filters. These filters, while reducing false alarms, may significantly decrease the dynamic response of the oximeter so that the true alarm may be delayed. Since, as noted, pulse oximeters are now being coupled with telemetry units for transmission of the oxygen saturation values to a central station, the transmission may be intermittent (for example to save battery power) with the central station updated only at predetermined intervals. This can result in an additional delay. Each of these delays can be additive and this can seriously reduce the remaining time available to hospital personnel after the alarm to reverse the respiratory arrest before the onset of the dual arrest state.

Scientists in the field of pulse oximetry have been working hard to improve patient monitoring. Much of the work has focused on the adverse effect induced by excessive false alarms on the timeliness of the response to a true alarm. In response, manufacturers have provided selectable delays with some pulse oximeters to reduce the number of false alarms, but the disclosed methods may reduce false alarms by producing a further delay in response to true alarms. U.S. Pat. No. 5,865,763 entitled, “Method and apparatus for nuisance alarm reductions” (the disclosure of which is incorporated by reference as if completely disclosed herein) shows one such selectable delay system and provides additional background for the present invention.

When monitored by the basic conventional hospital montage (which includes electrocardiogram, pulse oximetry, and chest wall impedance, EKG), the human physiologic system produces a large array of highly interactive time series outputs, the dynamic relational configurations of which have substantial relevance when monitored over both brief and long time intervals, and which can be used to generate improved alarm response.

Critical illness is one example of a complex dynamic timed process characterized by a plurality of interactive primary and compensatory outputs. When human physiologic stability is under threat, it is maintained by a complex array of interactive physiologic systems, which control the critical time dependent process of oxygen delivery to the organism. Each system (e.g. respiratory, cardiac or vascular) has multiple biochemical and/or mechanical controls, which operate together in a predictable manner to optimize oxygen delivery under conditions of threat. For example, a respiratory arrest (as with breath holding) causes a fall in heart rate to protect the heart muscle from the fall in oxygen. In addition to the basic control of a single system, other systems interact with the originally affected system to produce a predictable pattern of response.

Each system generally also has a plurality of predictable compensation responses to adjust for pathologic alteration or injury to the system and these responses interact between systems. For example, the development of infectious injury to the lung will generally result in an increase in respiratory rate to compensate for the loss of functional surface area. This increase in ventilation rate can then induce a synergistic increase in both stroke volume and heart rate.

Finally a pathologic process altering one system will generally also induce an alteration in one or more other systems and these processes are all time dependent. Sub acute or acute life threatening conditions such as sepsis, pulmonary embolism, or hemorrhage generally affect the systems in cascades or predictable sequences which may have a time course range from as little as 20 seconds or more than 72 hours. For example, the brief development of airway collapse induces a fall in oxygen saturation and a fall in heart rate. This may cause a compensatory hyperventilation response, which causes a rise in heart rate and all of this may occur in over as little as 20-30 seconds. Alternatively, the respiratory arrest progressive fall in oxygen coupled with a fall in heart rate may progress to the state of dual arrest. The progression to this state may be greatly accelerated by the presence of poor ventricular function, electrical or conduction instability, or coronary disease. An infection, on the other hand, has a more prolonged time course often over a course of 48-72 hours—inducing a rise in respiration rate, a rise in heart rate, and then a progressive fall in oxygen saturation and finally respiratory arrest and a terminal fall in heart rate. As effective physiology compensation becomes exhausted, the final event of respiratory failure and arrest can occur precipitously during the night (when hospital staffing is low) thereby surprising the hospital staff with, what appears to be a sudden respiratory arrest.

As discussed in detail below, the present inventor recognized that precipitous pathophysiologic catastrophic events such as respiratory arrest are generally preceded by at least a brief episode of instability, which falls within a range of definable patterns. He further recognized that both the instability and the catastrophic occurrence involves multiple interactive organ systems which produce definable relational patterns indicative of the occurrence which could then be exploited using a microprocessor to more timely recognize the adverse occurrence. Upon this realization the developed a system and method which provided signal integration and/or comparison of multiple signals, and in particular the patterns defined by multiple signals to define an alarm threshold, provided a better means to improve timely response to a catastrophic occurrences such as a respiratory arrest. According to the present invention, the recognition of combined deviation of at least one parameter in combination with a fall in oxygen saturation (especially if the other parameter is identified and/or confirmed by another method such as EKG) can be used to generate an earlier alarm and a more reliable alarm.

As discussed in U.S. Pat. No. 7,081,095, filed May 17, 2002 (which is incorporated by reference in its entirety, as if completely disclosed herein), during health and disease, organs have a basal state (such a diastole, or functional residual capacity) and a variation (a reciprocation), away from, and back to, that basic state. At the organ level a physiologic control system, which can be anatomic, electrical, or chemical attempts to maintain normal ranges of the basal state and induces return toward the basal state. At higher levels, a general basal state of the entire organism, and the interactive collective basal state of each organ, is generally maintained by a combination of interactive chemical, neural, and anatomic control. Upon variation from the basal state the physiologic control system will attempt to reverse the variation thereby producing reciprocation. The variation produces a compensatory response, which comprises a “companion reciprocation” which can be recognized by the processor and which improves the specificity of the recognition of the primary reciprocation. It can be seen that during very severe disease the attempt to achieve reversal may be unsuccessful so that reciprocations have the characteristic of being complete or incomplete. Interactive companion reciprocations operative from the cellular level to the organism level and exist across the entire range of time series scales and represent the fundamental link between the time series output of an organ or organism and the characterization of the operative control systems controlling that organ or organism, and the pathophysiologic process impacting the organ or organism.

As described in detail in provisional application Nos. 60/291,687 and 60/291,691 (the contents of each of which are incorporated by reference as if completely disclosed herein), the present inventor recognized that the onset of development of a catastrophic event represents the onset of a unique and critical relational time series of multiple signals (such as pulse rate, respiratory rate, and oxygen saturation) which is best considered as a relational process and tracked in close detail to generate a more specific output such as an alarm and so that the physician or nurse arriving to respond to the alarm can immediately be provided with an interpreted output of the evolution of all of the monitored interactive parameters upon the onset of the occurrence which generated the alarm. The value of a monitoring device as a early warning system is therefore direct and close function of its ability to timely alarm in response to the actual onset of the occurrence of a precipitous life threatening event rather than the timeliness of its response to the occurrence of one or more threshold breaches along a single parameter (such as oxygen saturation). This is distinct from traditional thinking with respect to oximetry (see U.S. Pat. No. 5,865,763) where the focus has been to define the alarm either as a simple function of the occurrence of one or more crossings of a particular threshold value (such as an oxygen saturation below 85-90%) or as a function of the occurrence of a cumulative magnitude of values below a simple threshold. In fact the controversy within the standards committee of the FDA for oximetry alarm guidelines has been focused not defining the relationship of the alarm response to the relational outputs of a catastrophic occurrence, but rather upon defining which one-dimensional alarm warning threshold value of oxygen saturation should be used (such as below 90% or below 85%). Such controversy misses the point, since, in the presence of precipitous catastrophic occurrence is not a given arbitrary threshold alarm value which is critical to functional survival but rather it is the value of time between the onset of the catastrophic event and the onset of the alarm which is best defined by the relational evolution of multiple parameters.

The present inventor recognized physicians, manufacturers, and hospitals have had a long unfulfilled need for an apparatus and method to alarm in response to the dynamic, real world pathophysiologic occurrences.

The present invention comprises a method and apparatus for providing an alarm based on a relational conformation of a plurality of time series and can include a pulse oximeter based microprocessor alarm system for the recognition of specific dynamic patterns of interaction between a plurality of corresponding and related time series, the system comprising a processor, the processor programmed to: process a first time series to identify a first primary threshold breach based on the first time series, process at least a second time series, compare at least a portion of the first time series to at least the second time series to produce a comparison result, identify a relationship between the first time series and the second time series to identify a relational threshold breach, output an alarm based on at least one of the primary and the relational threshold breaches. The first time series may be oxygen saturation and the second time series may be pulse. Alternatively, the first time series may be the oxygen saturation of arterial blood and the second time series may be the respiration rate and or amplitude or a derivative of both the rate and amplitude (as by chest wall impedance). The relational breach may be a fall in oxygen saturation coupled with a fall in pulse. Another relational breach may be a fall in oxygen saturation coupled to a rise in respiration rate.

In one embodiment, the physician can select from a menu, which signals are to be included in the alarm response so that the alarm is tailored to the patient. According to the present invention, the system includes the conventional threshold based alarms to provide a floor of protection with the relational alarm system of the present invention included to provide a more timely response upon the occurrence of instability or a precipitous life-threatening event such as respiratory arrest.

It is a purpose the present invention to provide a method and apparatus for providing dynamic alarm function of oximeters in response to precipitous catastrophic occurrences.

It is further a purpose of the present invention to provide a method and apparatus for promoting the sale of improved oximeters, which provide enhanced early warning characteristics, features, and functionality.

### BRIEF DESCRIPTION OF THE DRAWINGS

FIG. 1 shows a schematic of a relational alarm system according to embodiments of the present invention.

FIG. 2 shows a schematic of the processing method for analyzing a plurality of time series to provide earlier recognition of a pattern indicative of a particular a respiratory arrest according to embodiments of the present invention and defining the time onset of an alarm of a pulse oximeter.

FIG. 3 shows a schematic of one preferred embodiment of the oximeter testing apparatus.

FIG. 4 shows one processing method for testing of an oximetry system's ability to timely warn of a real world catastrophic occurrence using a respiratory arrest simulation.

FIG. 5 depicts a schematic of a processing system for outputting and/or taking action based on analysis of time-series processing in accordance with certain embodiments.

FIG. 6 is a schematic illustration of one embodiment of a portable minute volume indexing pulse oximeter according to certain embodiments.

FIG. 7 is a schematic illustration of a spirometer coupled to a pulse oximeter for generating a ventilation indexed pulse oximetry value according to certain embodiments.

FIG. 8 is a schematic diagram of a processor for coupling with a spirometer and a pulse oximeter for generating a ventilation indexed pulse oximetry value according to certain embodiments.

### DESCRIPTION OF THE PRESENTLY PREFERRED EMBODIMENTS

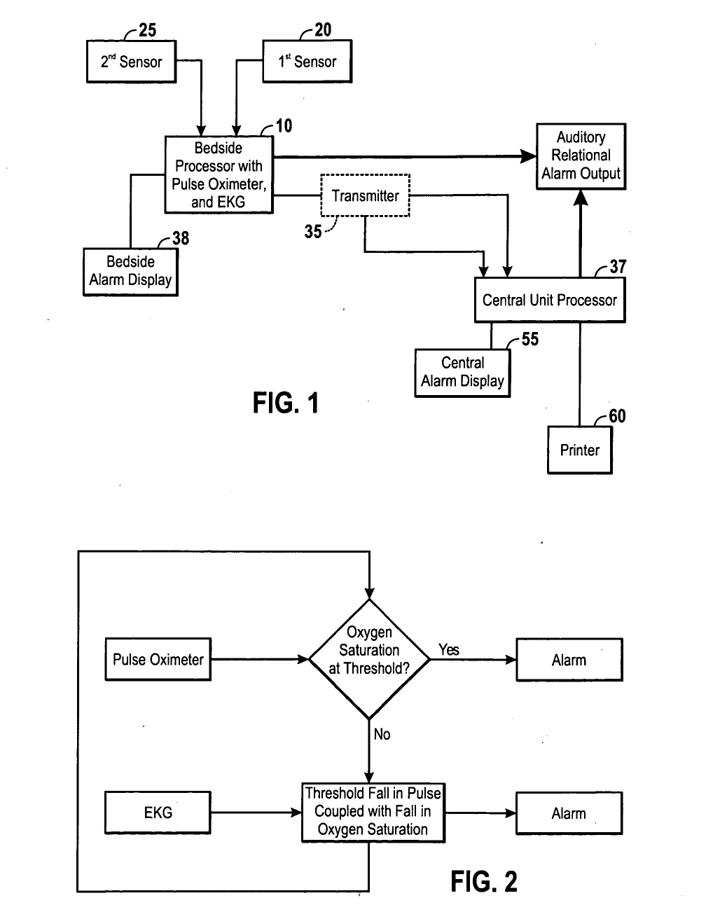

FIG. 1 shows a relational alarm system for real time detection of a broad range of patterns and instabilities (as described in the aforementioned co pending patent application). The system may include a portable bedside monitor 10, which may incorporate a pulse oximeter having at least a first sensor 20 and a electrocardiogram or other monitor including at least a second sensor 25. The system may include a transmitter 35 to a central processing unit 37. The bedside processor 10 may include an output screen 38, which provides the nurse with a bedside indication of the sensor output. The central unit 37 may include an output screen 55 and printer 60 for generating a hard copy for physician interpretation. The system may provide recognition and alarm of catastrophic occurrences based on analysis of relational outputs of a plurality of time series thereby allowing recognition of respiratory arrest, airway instability, and complications related to such instability, and pathophysiologic divergence.

FIG. 2 shows a processing method for the relational alarm system. In one embodiment the oxygen saturation may be monitored along with the pulse rate (which may be derived from another sensor so that the same artifact is less likely to affect both sensors). In an example, as is conventional, if the processor identifies a fall in oxygen saturation or pulse that meets threshold (for example 85% and 50 respectively), the alarm sounds. However according to the present invention, if on the other hand the primary threshold is not reached but a secondary threshold is reached (for example a fall of 5% within 45 seconds) and there is a combined fall in oxygen saturation in association with a concurrent fall in heart rate (for example a fall of 8 or more beats per minute within 45 seconds) wherein the fall in heart rate developed in relation to the fall in oxygen saturation (immediately prior to, as for example 30 seconds or less), at the same time, or immediately thereafter) the alarm sounds indicating a relational output suggestive of an adverse event and that relational output is provided for over reading by the hospital worker. Here, it can be seen upon the teaching of the present invention that in the presence of adverse relational outputs, delaying the alarm until the primary 85% threshold breach is reached may not be warranted.

Upon this teaching, those skilled in the art will recognize that many alternative relational outputs can be combined and analyzed along with oximetry (as by the time series analysis methods described in the aforementioned patent applications to the present inventor) to provide improved and more specific alarms and these are included within the scope of this teaching. For example, a menu system for identifying the relational parameters to be included in the alarm could be provided. These can be selected by the user or coded in advance as a set of alarm montages for application to a specific group of patients (e.g. based on the entered patient activity classification such as ambulatory, restless, quiet, comatose). The nurse could select specific relationships, which he or she desires for recognition and warning by the processor. According to the present invention the relational alarms could be customized to identify instability as well as a life-threatening occurrence. One example of instability warning would be the selection of an alarm triggered by a relational pattern identified by the processor defined by a fall in oxygen saturation of X (say greater than or equal to 8%) coupled with a rise in respiration rate of Y (say 50% or more) lasting for at least Z (say 5-10 minutes). A relational pattern of signals falling within this range is highly indicative of respiratory instability. Accordingly, it is one of the express purposes of embodiments of the present invention to provide for physicians and nurses, using these different warning relationships and patterns, considerably more functional and discretionary surveillance over different groups of patients.

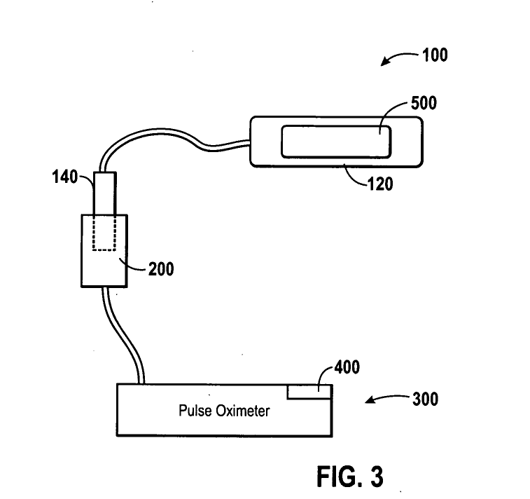

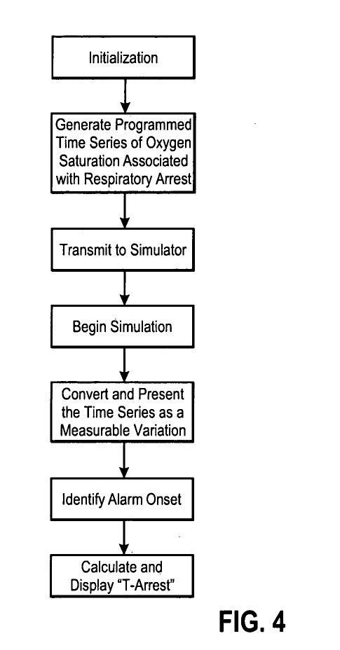

FIG. 3 shows one embodiment of the oximeter testing apparatus 10, which can be used with a conventional oximeter or with a relational alarm based system. The testing system may include a processor 12 in connection with a dynamic simulator 14 (which can be shaped to be received into a conventional finger probe) for interfacing with the probe 20 of an oximeter 30. A processor 12 may control the output of the simulator 14 according to the process of FIG. 4. The processor 12 may be programmed to simulate the time series of arterial oxygen saturation and one or more additional parameters, which are generated in association with the occurrence of a precipitous apnea or respiratory arrest as from functional residual capacity. The programmed time series can be predicted by known formulas, or the time series of oxygen saturation and pulse (if also included) can be defined by or calculated from published clinical trials. In this embodiment, the simulated occurrence is a complete respiratory arrest occurring precipitously with the lung volume at functional residual capacity and at room air (21% oxygen) at the time of onset of the arrest. The dynamic oxygen saturation time series simulation for this occurrence can be, for example, the known delay and subsequent shape and slope of fall of the oxygen saturation time series (as can be calculated using known formulas for conversion of partial pressure of oxygen time series to oxygen saturation time series) as determined with breath holding clinical trials. (An example of such a clinical trial was published in Chest in 1996 entitled. “Arterial Blood Gas Changes during Breath-Holding from Functional Residual Capacity” by Sasse et. al.) The Severinghaus equation, or other formula, can be used to calculate the time series of oxygen saturations from the PaO2, PH, and PaCO2 values from clinical trials such as the one above which publish only the blood gas values. This can be coupled with the know range of falls in pulse if a relational monitor is being tested.

The processor may be programmed to output a baseline saturation (such as 97-100%) and then to begin (automatically or on command) the simulation of the precipitous respiratory arrest. The processor sets the clock at the onset of the simulated respiratory arrest. The processor may be further programmed to then output a time series of oxygen saturation values with the predicted real time fall in oxygen saturation according to that predicted for such an event to the simulator for presentation to the probe. The time series of oxygen saturation values from the processor mirrors those calculated for the article noted or can be another time series, which mirrors the dynamic changes of oxygen saturation during a particular type of critical illness. In one embodiment, an audio sensor is provided in connection with the processor, the sensor is responsive to the high frequency sound produced by the oximeter alarm. Upon the occurrence of the alarm, the sensor outputs an indication of the alarm occurrence to the processor, which records the time of onset of the alarm occurrence. Alternatively the time of onset of the alarm occurrence can be input by hand or automatically by connecting the processor of the oximeter and the processor of the testing device. The difference between the time of onset of the simulation of the respiratory arrest and onset of the alarm occurrence is calculated by the processor and outputted as the “TA” or “T-Arrest” value (given in seconds).

The “T-Arrest” may be defined as: The time from the onset of a standardized respiratory arrest simulation to the onset of the alarm. This is a single number used to provide evidence of the performance of the oximeter as an early warning device. In one embodiment the T-Arrest is determined during motion and is given as the “TAM” or the “T-Arrest (Motion)”. According to present embodiments, oximeters will be marketed by publishing the TA and the TAM, which (like the simple horsepower value in an automobile) may be used to determine performance. It is anticipated that present embodiments may be used by hospitals to test their existing oximeters and to test new devices before purchase. In one embodiment, the audio sensor has a threshold value equal to a decibel level, which is readily heard and recognized by a human ear.

The oximeter testing system can be incorporated into the oximeter as a self-testing method or process, which may, for example be initiated by keying in a “simulate physiology” button on the oximeter, which will then run an internal simulation and report the T-arrest. The menu could include a range of simulations, for example a time series with the alveolar oxygen at 21% at the onset of arrest in an adult, and then another with the alveolar oxygen at 40%, and a third which simulates a cardiac arrest with loss of the plethesmographic pulse, a fourth which simulates a respiratory arrest in a child or neonate. These internal simulations can be used for teaching purposes and can combine additional inputs such as pulse rate to test relational alarm features if provided. In one embodiment, the menu can include the selection of the inspired oxygen level and the occurrence of a specific level of ventilation (minute ventilation) and dead space.

Alternative configurations of the simulator can include the addition of a transmitter for transmitting the simulated oxygen saturation output from the processor to a receiver of a separate simulator for interfacing with the probe of an oximeter at a remote location such as a patient's room. Hospitals can position a given oximeter at a bedside and the alarm testing device at the nurses station and then initiate the testing sequence to determine if the alarm will be recognized by the nurses at the station and how long after an arrest they will hear the alarm with a given oximeter (if at all). The processor can include an audio sensor 40 capable of providing an output indicative of the occurrence of an audio alarm at a specified decibel level. The processor can also include an input indicator for manually indicating when action has been taken (e.g. the time at which the beside has been reached with the ventilation equipment in hand.)

In operation the probe 200 may be connected to the simulator 140 of the alarm testing system 100, as in a patient's room. The operator may then input to initiate the simulation. The simulation may then be initiated. The processor may transmit the simulated saturation time series based on the time series predicted form the occurrence of a respiratory arrest to the simulator 140 as previously discussed and record the time of onset of the simulation. The simulator 140 presents to the probe 200 a measurable timed variation indicative of the saturation time series. When the oximeter 300 generates an auditory alarm at the specified decibel level in response to the outputs of the simulator 140, to the site of the processor 120, the time of onset of the alarm may be recorded and the difference between the time of onset of the arrest simulation and the time of onset of the alarm may be calculated and presented on the display 500. In some situations the oximeter under test may also include a transmitter, which transmits the oxygen saturation output back to a central nursing station where the alarm will sound. Since such transmissions also may include averaging intervals or long intervals (20 seconds) between sample transmissions, the effect of this delay can also be determined by this system 100. The system 100 can be used for spot (surprise) checks of the alarm response times in a nursing ward where all of the factors: patient physiologic delay, signal processing delay, transmission delay, alarm output delay, personnel response delay, and the delay associated with the time to reach the bedside (where the nurse inputs the endpoint), are automatically included providing a true index of the effective hospital response to an actual alarm indicative of a life threatening event.

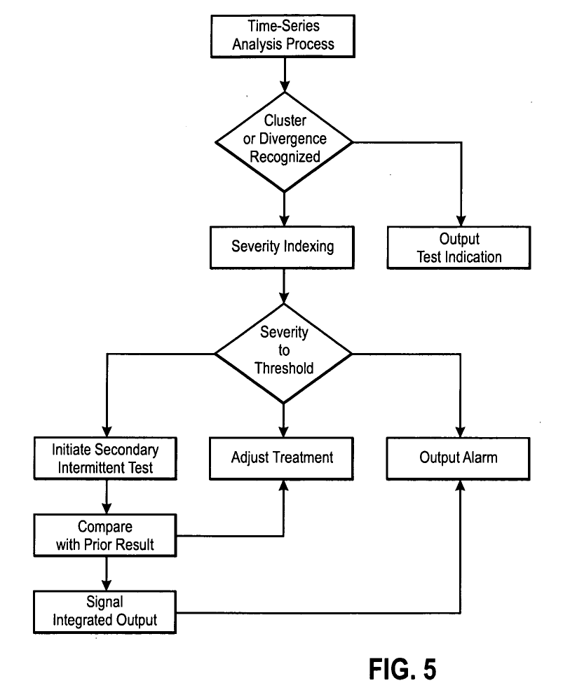

FIG. 5 depicts a schematic of a processing system for outputting and/or taking action based on analysis of time-series processing in accordance with certain embodiments of the present technique.

The “Time-Series Analysis Process” block of FIG. 5 may represent a system component related to analyzing one or more time series. In accordance with present embodiments, time-series analysis can be utilized to analyze multiple time-series of parameters generated by a patient in the assessment of disease. For example, a time-series of a patient's heart rate data may be analyzed. Other examples of parameters that may be analyzed include oxygen saturation, chest wall impedance, pulse rate, and blood pressure.

The system of FIG. 5 also includes a block titled “Cluster or Divergence Recognized,” which may represent a system component configured to recognize a cluster or divergence of one or more time-series. Recognition of a cluster may be achieved by analyzing spatial and/or temporal relationships between different portions of a waveform. For example, a cluster may contain a high count of apneas with specified identifying features or patterns that occur within a short time interval along the waveform (such as 3 or more apneas within about 5-10 minutes). With regard to divergence, in accordance with present embodiments, a change in configuration of a multi-signal time-series can be used to trigger addition of one or more signals to the multi-signal time-series to identify whether or not physiological divergence is occurring with respect to the new, less frequently sampled signal.

By way of example, a processor in accordance with present embodiments may identify a significant rise in heart rate (e.g., a 25% rise and at least 15 beats per minute) over a period of 5 to 20 minutes. In view of such a rise, a monitor may automatically cause a measurement of blood pressure to be immediately taken. The processor may compare an output of the monitor to a previously recorded value and, if a significant fall in blood pressure (such as a fall in systolic of 15% and more) is identified in association with the identified rise in heart rate that triggered the test, a textual warning may be provided indicating that the patient is experiencing pathophysiologic divergence with respect to heart rate and blood pressure. The “Output Text Indication” block in FIG. 5 may represent a system component for providing such a warning.

In addition, the system may include features configured to index the severity of a recognized cluster or divergence, and make a severity to threshold determination. Such system components may be represented by the “Severity Indexing” and the “Severity to Threshold” blocks illustrated in FIG. 5. In accordance with present embodiments, the severity to threshold determination may result in an alarm. For example, mild clustering may result in outputting a single bar on a bar indicator, while a more severe clustering may result in generation of a larger warning. Such warnings may be represented by the “Output Alarm” box in FIG. 5.

The severity to threshold determination may also lead to adjusting a treatment, as indicated by the “Adjust Treatment” block. Further, the determination may also lead to initiation of a secondary intermittent test, as shown by the “Initiate Secondary Intermittent Test” block. The result of the secondary test may be compared to the prior results, as represented by the “Compare with Prior Result” block, which may also lead to a treatment adjustment. In addition, the results of the comparison may be combined into a signal integrated output, as illustrated by the “Signal Integrated Output” block, which may activate an alarm.

The following are examples of clinically useful indices which can be calculated by the described embodiment (the corresponding time interval for each of these indices may be adjusted for the delay in oxygen uptake and transmission into oxygen saturation data by the pulse oximeter, as is known in the art):

1. The saturation to ventilation index

(SaO2.sub.ta−84)/Ve.sub.t

Where; Vet=the average minute ventilation during a time interval and,

SaO2t=the average arterial saturation during the corresponding time interval.

The higher this index, the greater the probability of a hypoventilation disorder and attendant sleep disordered breathing (as shown, this index is only for saturations of 85 or above).

2. The delta saturation to delta ventilation index

dSaO2.sub.ta/dVe.sub.t(change in % saturation/change in minute ventilation)

Where; Ve.sub.t=the average change in minute ventilation during a time interval (either increase or decrease) and,

SaO2.sub.ta=the average change in arterial saturation during the corresponding time interval.

Patients with intrinsic lung disease have a lower index than normal. This index helps differentiate whether a low baseline saturation is due to hypoventilation or intrinsic lung disease.

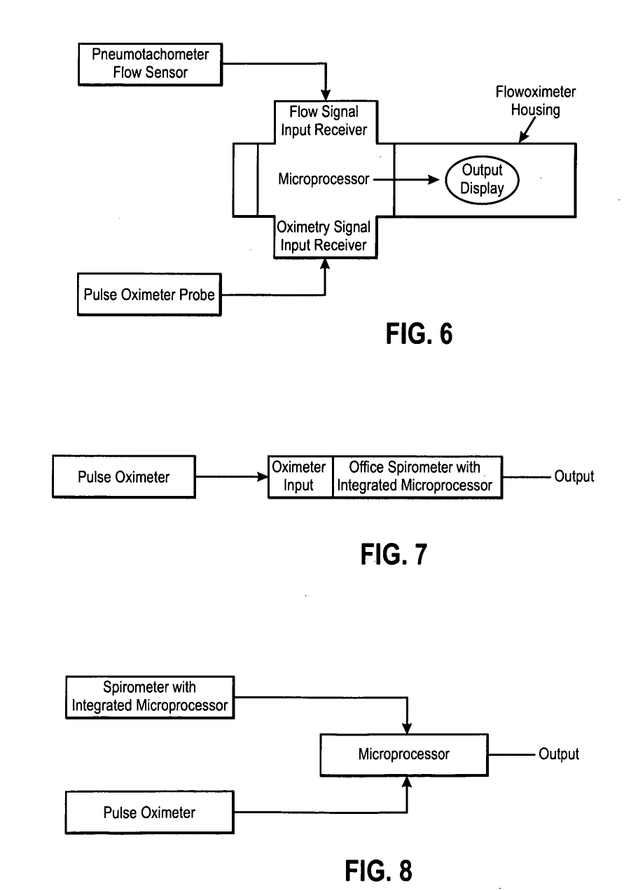

FIG. 6 illustrates a compact single unit configuration which may be referred to as “portable flowoximeter” which allows portable determination of the above parameters. This configuration may be achieved by:

1. Combining an oximeter and spirometer into a single compact casing which can be easily carried to the bedside.

2. Attaching a pulse oximeter probe and a flow sensor to the integrated pulse oximeter.

3. Using the microprocessor, integrating the timed oximetry signal and timed exhaled (or inhaled) gas flow (an adjustment may be made for the oximetry signal delay as described in U.S. Pat. No. 6,223,064, for example.)

4. Providing output of oxygen saturation indexed for the timed or averaged exhaled volume.

FIG. 7 illustrates an embodiment for office use. The configuration and operation may be achieved by:

1. Providing an oximetry signal input receiver as part of an office spirometer.

2. Using the microprocessor, integrating the timed oximetry and timed flow (an adjustment may be made for the oximetry signal delay as described in U.S. Pat. No. 6,223,064, for example).

3. Providing output of oxygen saturation indexed for the timed or averaged exhaled volume.

FIG. 8 illustrates an embodiment for use with existing oximeters and spirometers having output jacks which can be accessed for connection to a central microprocessor. The configuration and operation may be achieved by:

1. Connecting the output of an oximeter and the output of a spirometer to a microprocessor.

2. Using the microprocessor, integrating the timed oximetry flow (an adjustment may be made for the oximetry signal delay as described in U.S. Pat. No. 6,223,064, for example).

3. Providing output of oxygen saturation indexed for the timed or averaged exhaled volume.

The sleep apnea diagnostic system, as described in U.S. Pat. No. 6,223,064, can be used to determine the severity of sleep apnea. Studies have demonstrated that the standard “apnea hypopnea index” which is calculated by counting the number of apneas and hypopneas and dividing by the number of hours of sleep is a poor indicator of disease severity. There has long been a need for a new method to assess severity. Indeed, studies sponsored by the National Institute of Health may identify the validity of the apnea hypopnea index and to identify a valid signal of disease severity. As discussed in U.S. Pat. No. 6,223,064, the cluster characteristics can be used to define severity. The present inventor has discovered a system and method, which can be used to enhance the determination of disease severity. In the present embodiment, this system and method may determine a value indicative of the sufficiency of recovery associated with sequential apneas and uses at least this value to define the disease severity in sleep apnea.

It has been long believed that the number and duration of apneas determined the severity of disease. This basic concept seems intuitive. Indeed, if apneas are the detrimental events then it seems rational that the number and duration of the apneas would define severity. Perhaps because the severity issue seems so straightforward, the concept of severity as a function of enumeration of apneas has been promulgated for decades and represents the standard of severity assessment in modern sleep medicine. However, the present inventor noted that with respect to breath holding that severity cannot be determined by the number and longevity of the breath holdings or by the magnitude of arousals or oxygen desaturation associated with the breath holdings, but rather is uniquely dependent on the sufficiency of the recovery interval between breathholds. This is due to the fact that mammals (including humans) have an oxygen storage mechanism to protect against the stress of breatholding but this storage mechanism is readily depleted and must be repleted before the next breathhold. This means that, with respect to apnea, there is a unique and critical relationship between cardiovascular stress associated with the sequential breathholds which naturally occur as a part of a self propagating apnea cluster and the recovery interval between breathholds. Indeed, this “sufficiency of recovery” between apneas is defined by a critical interaction between the number of breathholds occurring in sequence, the longevity of the breatholds, and the recovery time between the breatholds. As discussed in U.S. Pat. No. 6,223,064, these relationships are provided by analyzing the apnea cluster waveform. The present inventor has proposed that the diving seal provides a reasonable analogy. The seal can dive very often and long without cardiovascular compromise as long as the animal has sufficient free breathing recovery intervals above water between dives. When this interval is limited the seal faces a serious cardiovascular threat if the dives are frequent and prolonged. Indeed, in the wild, the need for sufficient recovery interval is exploited in the interest of predation. It is this unique relationship between severity and the sufficiency of recovery between sequential apneas which the present inventor has utilized to provide a new system and method to determine the severity of sleep apnea.

The present inventor designed a system and method of evaluating a patient with sleep apnea. The method includes identifying a plurality of sequential apneas, determining a value indicative of the sufficiency of recovery between the apneas, and determining the severity of sleep apnea based at least on that value. The value can be a measurement such as the time interval or another measurement such as the relative amount of gas exhaled or inhaled from the mouth or nose between apneas. Both the interval time and the relative amount of gas exhaled can be used in combination and another value indicative of the sufficiency of recovery can be calculated using this combination as for example the product of the time and a measurement of the relative gas exhaled. The exhaled gas can be measured directly or inferred relative to a baseline using a flow sensor (as is known in the art). Another value indicative of the sufficiency of recovery is a measurement indicative of an oxygen saturation between apneas which can for example be expressed as the average oxygen saturation of the recovery interval. A present embodiment can include the steps of monitoring a patient to produce at least one timed waveform of at least one physiologic parameter. The physiologic parameters can include for example arterial oxygen saturation, the flow of gas at the nose and or mouth, (as can be measured by a thermister or a carbondioxide monitor as is known in the art), or chest wall movement. Then, the step of identifying along the waveform a first waveform variation indicative of an apnea, then identifying along the waveform a second waveform variation indicative of another apnea, then determining (as for example by measuring or calculating) the interval intermediate at least one portion of the first waveform variation and at least one portion of the second waveform, and assessing the severity of sleep apnea based on at least that determination.

An example of a waveform variation indicative of apnea is described in U.S. Pat. No. 6,223,064 for the oximetry waveform and can comprise a coupled desaturation and resaturation having characteristic slopes and occurring within a desaturation cluster. One portion of the first waveform variation may correspond to one portion of the second waveform variation, and one portion of the first waveform variation may be substantially the last portion of the first waveform variation but the interval can extend into each waveform variation as is described below for the interval termed the oxygen repletion interval where a portion of the second waveform variation is incorporated into the intervening interval. One portion of the second waveform variation can be substantially the first portion of the second waveform variation.

The method can include identifying at least one cluster of waveform variations indicative of a corresponding cluster of apneas wherein the severity assessment or determining is based on the position of the waveform variations within the cluster relative to other waveform variations within the cluster. Alternatively or in combination a spatial cluster waveform pattern indicative of a spatial pattern of a corresponding cluster of apneas can be used to determine the spatial relationships of the waveform variations within the cluster waveform pattern to determine the severity of sleep apnea.

The device for determining the severity of sleep apnea can comprise a monitor, such as an oximeter or flow sensor, capable of generating a signal indicative of at least one physiologic parameter and a processor (such as an integrated computer or a separate lap top computer) capable of processing the signal. The processor can operate to generate a timed waveform of the parameter and to identify a plurality of sequential waveform variations indicative of a corresponding plurality of sequential apneas. The sequential waveform variations may have temporal and spatial relationships between the waveform variations and along the waveform (as was discussed at length in U.S. Pat. No. 6,223,064, for example). The processor further can operate to determine at least one of the temporal and the spatial relationships and to display the result or determination so that the determination can be used to assess the severity of sleep apnea.

The method of determining the severity of sleep apnea can comprise the steps of identifying a plurality of sequential apneas having a spatial relationship to each other, determining the spatial relationship, and defining the severity of sleep apnea based on at least the determination. The spatial relationship can be defined by an object oriented method as discussed in U.S. Pat. No. 6,223,064 or by other known graphical methods which include pattern recognition or graphical event recognition.

Alternatively or in combination with the methods noted above, a method of determining the severity of sleep apnea can comprise steps of identifying a plurality of sequential apneas having a temporal relationship to each other, determining the temporal relationship, and defining the severity of sleep apnea based on at least the determination. The temporal relationship can be defined by an object oriented method as discussed in U.S. Pat. No. 6,223,064, for example, or by other known methods such as frequency analysis or graphical event recognition.

One embodiment of a method to define the severity of sleep apnea is as follows:

Define a Desaturation Object as including two component objects:
1. An Initial Limb defined as that portion of the desaturation above a specific threshold saturation (e.g. to a sat. of 80%) or as a percentage of the total fall in saturation (e.g. Forty percent of the total fall).
2. A Terminal Limb defined as that portion of the desaturation remaining after the Initial Limb.
Define a Positive Oscillation as a Resaturation followed by a Desaturation within a specific interval of less than 60 seconds.
Within each cluster, define a Repletion Interval as an object including a Resaturation, a Plateau (the plateau may be absent) and an Initial Limb within a Positive Oscillation.
It should be noted that by extending this calculation through the initial limb, this calculation takes into account the effect of an increased initial portion of the subsequent desaturation slope on the oxygen availability during the recovery interval.
Calculate the average oxygen saturation during each Object as: ##EQU1##
Where: i=1 is the initial sample of the object, and i=1 is the final sample, and .DELTA.tau. is the time interval between samples.

With the object oriented program previously described the average oxygen saturation can comprise a characteristic of an object and, as such, can be easily compared and plotted (for example, against duration of each object). This can for example, be applied to the repletion intervals within a given cluster. When plotted in this manner with saturation on the y-axis and time on the x-axis a grouping of repletion intervals within the left lower quadrant of the plot is indicative of cluster with a low mean or median recovery interval in association with a low mean or median oxygen saturation during the recovery interval. This is generally indicative of more severe sleep apnea but the indicator of severity is further enhanced and improved by incorporating the duration of the adjacent apneas such as the immediately bracketing apneas into the plot or calculation. The processor-based system described in U.S. Pat. No. 6,223,064 can be used to assess and graphically present a severity analysis and to calculate a severity index, which can be, derived as follows in an embodiment.

1. PROGRAM—identifies the object cluster as defined in U.S. Pat. No. 6,223,064.
2. PROGRAM—calculates the following for each cluster object:

1. Mean and median saturation (iterates through oxygen saturation values).

2. Mean and median apnea and cluster duration.

3. Mean and median recovery interval (calculated as end of the nadir to onset of next desaturation) and calculate avg. sat for each recovery interval and mean and median avg. saturations for said recovery intervals.

4. Mean and median maximum oxygen repletion interval (as mean recovery interval +40 percent of the next desaturation event) and calculate the avg. saturation and mean and median avg. saturations for this interval.

5. Plot the distribution of the duration for each repletion interval (x-axis) with either the average saturation for each repletion interval (y-axis) or the average duration of the apneas before and after each repletion interval (y-axis) (For example the durations of the two apneas immediately before and two apneas immediately after the repletion interval divided by 4). This plot is performed for each cluster object and also performed as one plot in aggregate for all clusters.

6. Calculate the mean and median saturation of non clustered recording.

7. Calculate the time in minutes below 90%, 85%, 80%, and 70% during cluster objects and during recording time wherein said objects are not present. Plot this as a comparison bar graph side by side (non clustered bar for 90% adjacent clustered bar for 90% etc.) where the y axis is percent of total recording total recording time and again on another graph where the y axis is time in minutes (the length of the y axis is equal to the total recording time).

8. Plot total cluster time and total non cluster time on a separate graph.

9. Calculate a value of the sufficiency of recovery as a sleep apnea severity index termed the “Oxygen Repletion Index” (ORI) as the product of Oxygen Saturation minus 80 and the repletion interval. The ORI is given in “Saturation Seconds”. This can be calculated for each recovery interval and as a mean or median value for each cluster or portion of a cluster (such as a portion of a cluster having a greater apnea duration or a greater magnitude of desaturation) or for the entire night.

10. Calculated another value of the sufficiency of recovery as another sleep apnea severity index “Apnea Recovery Index” (ARI) as the quotient of the ORI and the mean duration of the apneas immediately bracketing each recovery interval. The ARI is given in “Saturation Seconds per Minute of Apnea”. This also can be calculated for each recovery interval and/or as a mean or median value for each cluster or portion of a cluster (such as a portion of a cluster having a greater apnea duration or a greater magnitude of desaturation) or for the entire night.

As noted, these sleep apnea severity indices can be calculated for the entire night as with the average index but may be visually presented to provide a more comprehensive picture of the heterogeneity of severity during a nights study. For this purpose the indices can be plotted for each cluster with each cluster as a new bar graph along a timed x-axis. The bar vertically presents the ORI with a zero line through the center. The entire study time may be plotted along the x-axis so that the width of the bar represents the duration of each consecutive cluster and the relative portion of the night spent without clustering is readily visible between the bars thereby allowing a straightforward visual presentation of the multiple parameters defining severity as discussed in U.S. Pat. No. 6,223,064.

Illustrative Examples of ORI and ARI calculations:

A mean repletion interval saturation of 90% and a Repletion interval of 20 seconds generates an ORI of 200 saturation seconds. If the bracketing apneas have a mean duration of two minutes this generates an ARI of 100 saturation seconds per minute of apnea indicating mild severity.

A mean repletion interval saturation of 85% and a Repletion interval of 20 seconds and a mean bracketing apnea duration of two minutes generates an ARI of 50 saturation seconds per minute of apnea indicating a greater degree of severity.

A mean repletion interval saturation of 82% and a Repletion interval of 20 seconds and a mean bracketing apnea duration of two minutes generates an ARI of 20 saturation seconds per minute of apnea indicating very severe disease.

A mean saturation of the repletion interval saturation of 78% and a Repletion interval of 20 seconds and a mean bracketing apnea duration of two minutes generates an ARI of −80 saturation seconds per minute of apnea. It should be noted here that in the event that the ORI is a negative number the mean duration of the bracketing apneas is multiplied times the ORI to generate the ARI. This level of ARI may be indicative of profound, life threatening severity.

It should be noted that saturations below 80% will generate a negative ORI. A lower negative ORI associated with increasing repletion times accounts for the fact that a long repletion time actually reflects greater severity when the mean saturation of the repletion interval is profoundly decreased since this may indicate profound disease.

Alternatively, the index can be based on the recovery interval time and the duration of the bracketing apneas without consideration of the oxygen saturation. These basic severity indices may be given in seconds per minute of apnea. Such severity indices are easy for physicians to understand. For the purpose of defining the relative potential for reduced oxygen delivery in the presence of critical vascular stenosis these indices can be weighted to maximize the effect of lower saturation on oxygen delivery during the repletion interval. Additional weighting can be provided to incorporate the average duration of the clusters in combination with the mean recovery interval within clusters which may be another important value indicating sleep apnea severity.

It is clear that alternative severity indices can be provided. For example, the number of breaths exhaled or inhaled between apneas can be used and indexed in combination with the mean duration of the bracketing apneas. Also, a wide range of alternative aggregate severity indices of obstructive sleep apnea can be provided utilizing the system and method of the present invention which incorporate measurements or identification of events or deflections along the waveform which correlate with the sufficiency of the recovery intervals. Such severity indices can include in combination with the repletion intervals, or recovery intervals, the enumeration or frequency evaluation of identified events or measurements indicative of apnea along the waveform or can include waveform pattern identification which provides for the identification of grouped or closely spaced waveform deflections which correlate with grouped or closely spaced apneas or apnea clusters having limited recovery intervals. As has been shown by this teaching, with such a grouping, the limitation of the recovery interval may be inferred, for example, by a particular waveform pattern of tightly grouped waveforms of high amplitude deflections.

In one embodiment, severity is defined as inversely proportional to the duration of the repletion intervals and the oxygen saturation of the repletion interval within the clusters and directly proportional to the duration of the apneas within the cluster. In addition, the number of breaths, the relative magnitude of the breaths, the slope of the initial 50% of the descending limbs of the desaturations within the cluster, and the duration of the clusters may all be incorporated to produce an aggregate index. Within the scope of this teaching, alternative intervals can be used in place of the illustrative repletion intervals and recovery intervals described herein and further additional intervals may be learned during the application of this teaching to clinical practice.

It will be evident to those skilled in the art that many additional modifications may be made, and these are included within the scope of the invention.

1. A pulse oximeter based system for the recognition of patterns in a plurality of time series, the system comprising a processor programmed to:
process a first time series to identify a first primary threshold breach based on the first time series;
process a second time series;
compare at least a portion of the first time series to at least a portion of the second time series to produce a comparison result;
identify a relational threshold breach based at least in part on the comparison result; and
output an alarm based on at least one of the primary and the relational threshold breaches.

2. An oximeter testing system for testing an oximeter, the system comprising:
a processor programmed to generate an output indicative of a time series of oxygen saturation values, the output simulating a time series of oxygen saturation values associated with an occurrence of an adverse clinical event, and programmed to calculate a difference between a time of onset of the occurrence and a time of onset of the alarm; and
an oxygen saturation simulator for presenting the output to the oximeter to determine a delay between the time of onset of the occurrence and the time of onset of the alarm.

3. The system of claim 2, wherein the output is simulating a time series of oxygen saturation values associated with a respiratory arrest.

4. The system of claim 2, wherein the output is simulating a time series of oxygen saturation values associated with a respiratory arrest occurring with a lung volume at functional residual capacity at the time of onset of the occurrence.

5. The system of claim 2, wherein the output is simulating a time series of oxygen saturation values associated with a respiratory arrest occurring with an alveolar oxygen concentration at about 21% at the time of onset of the occurrence.

6. The system of claim 2, wherein the processor is programmed to identify the time of onset of the alarm.

7. The system of claim 2, wherein the processor is programmed to measure the difference between the time of onset of the occurrence and the time of onset of the alarm.

8. The system of claim 2, wherein the processor is programmed to output an indication of the difference.

9. The system of claim 2, wherein the processor is programmed to output the difference.

10. The system of claim 2, wherein the oximeter includes a probe configured to interface with the oxygen saturation simulator via a member, the member configured to present a secondary dynamic variation simulating a primary dynamic variation in oxygen saturation, the secondary dynamic variation being based on the output, wherein the processor is configured to present the output to the member and the member is configured to present the dynamic variation to the probe.

11. The system of claim 2, comprising an audio sensor configured to automatically detect the onset of the alarm.

12. A method of processing data comprising a plurality of time series, the method comprising:
searching a first one of the plurality of time series for data corresponding to a primary threshold breach;
producing an alarm output if data corresponding to the primary threshold breach is discovered;
searching the first one of the plurality of time series and a second one of the plurality of time series for data corresponding to a relationship between the first one of the plurality of time series and the second one of the plurality of time series;
searching data corresponding to the relationship for data corresponding to a relational threshold breach; and
producing an output indicative of the relational threshold breach if data corresponding to the relational threshold breach is discovered.

13. The method of claim 12, wherein the output indicative of the relational threshold breach comprises an alarm.

14. The method of claim 12, wherein the first one of the plurality of time series comprises data corresponding to a physiologic parameter of a patient.

15. The method of claim 12, wherein the second one of the plurality of time series comprises data corresponding to a physiologic parameter of a patient.

16. The method of claim 12, wherein the first one of the plurality of time series corresponds to oxygen saturation.

17. The method of claim 12, wherein the second one of the plurality of time series corresponds to timed ventilation.

18. The method of claim 12, wherein the second one of the plurality of time series is indicative of ventilation timing.

19. The method of claim 12, wherein the second one of the plurality of time series corresponds to a respiratory rate.

20. The method of claim 12, wherein the second one of the plurality of time series corresponds to a respiratory amplitude.

21. The method of claim 12, wherein the second one of the plurality of time series corresponds to a magnitude of timed ventilation.

22. The method of claim 12, wherein the relationship comprises at least one pattern relationship.

23. The method of claim 22, wherein the pattern relationship is a generally downward sloping oxygen saturation.

24. The method of claim 12, wherein the relationship comprises at least one trending relationship.

25. The method of claim 12, wherein the relationship comprises a general fall in oxygen saturation coupled to a general rise in respiration rate.

26. The method of claim 12, wherein the relationship is a derivative of both a parameter rate and a parameter amplitude.

27. The method of claim 12, wherein the relationship comprises an indication of ventilation by chest wall impedance.

28. A method of processing data comprising a plurality of time series, the method comprising:
searching a first one of the plurality of time series for data corresponding to a primary threshold breach;
searching the first one of the plurality of time series and a second one of the plurality of time series for data corresponding to a relationship between the first one of the plurality of time series and the second one of the plurality of time series, the relationship being independent of the primary threshold breach;
searching data corresponding to the relationship for data corresponding to a relational threshold breach; and
producing an output indicative of at least one of the primary threshold breach and the relational threshold breach if data corresponding to at least one of the breaches is discovered.

29. The method of claim 28, wherein the output comprises an alarm.

30. The method of claim 28, wherein the first one of the plurality of time series comprises data corresponding to a physiologic parameter of a patient.

31. The method of claim 28, wherein the second one of the plurality of time series comprises data corresponding to a physiologic parameter of a patient.

32. The method of claim 28, wherein the first one of the plurality of time series corresponds to oxygen saturation.

33. The method of claim 28, wherein the second one of the plurality of time series corresponds to timed ventilation.

34. The method of claim 28, wherein the second one of the plurality of time series is indicative of ventilation timing.

35. The method of claim 28, wherein the second one of the plurality of time series corresponds to a respiratory rate.

36. The method of claim 28, wherein the second one of the plurality of time series corresponds to a respiratory amplitude.

37. The method of claim 28, wherein the second one of the plurality of time series corresponds to a derivative of both a respiratory rate and a respiratory amplitude.

38. The method of claim 28, wherein the second one of the plurality of time series corresponds to a magnitude of timed ventilation.

39. The method of claim 28, wherein the relationship comprises at least one pattern relationship.

40. The method of claim 28, wherein the relationship comprises at least one trending relationship.

41. The method of claim 28, wherein the relationship comprises a general fall in oxygen saturation coupled to a general rise in respiration rate.

42. The method of claim 28, wherein the relationship is a derivative of both a parameter rate and a parameter amplitude.

43. The method of claim 28, wherein the relationship comprises an indication of ventilation by chest wall impedance.
